SEQUENCE LISTING (37 sequences)

SEQ ID NO: 1 - DNA, 10 bp, synthetic construct
  source 1..10: mol_type = other DNA
agcagctgct

SEQ ID NO: 2 - DNA, 10 bp, synthetic construct
  source 1..10: mol_type = other DNA
agccgctgct

SEQ ID NO: 3 - DNA, 10 bp, synthetic construct
  source 1..10: mol_type = other DNA
acaagctgct

SEQ ID NO: 4 - DNA, 10 bp, synthetic construct
  source 1..10: mol_type = other DNA
ctaaaaatag

SEQ ID NO: 5 - DNA, 10 bp, synthetic construct
  source 1..10: mol_type = other DNA
ctaaaattag

SEQ ID NO: 6 - DNA, 10 bp, synthetic construct
  source 1..10: mol_type = other DNA
ctaaatttag

SEQ ID NO: 7 - DNA, 20 bp, unidentified
  source 1..20: mol_type = genomic DNA; note = Adeno-associated virus serotype 2
ctccatcactaggggttcct

SEQ ID NO: 8 - DNA, 143 bp, unidentified
  source 1..143: mol_type = genomic DNA; note = Adeno-associated virus serotype 1
ttgcccactccctctctgcgcgctcgctcgctcggtggggcctgcggaccaaaggtccgcagacggcagaggtctcctctgccggccccaccgagcgagcgagcgcgcagagagggagtgggcaactccatcactaggggtaa

SEQ ID NO: 9 - DNA, 145 bp, unidentified
  source 1..145: mol_type = genomic DNA; note = Adeno-associated virus serotype 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 10 - DNA, 146 bp, unidentified
  source 1..146: mol_type = genomic DNA; note = Adeno-associated virus serotype 3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 11 - DNA, 146 bp, unidentified
  source 1..146: mol_type = genomic DNA; note = Adeno-associated virus serotype 4
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc

SEQ ID NO: 12 - DNA, 167 bp, unidentified
  source 1..167: mol_type = genomic DNA; note = Adeno-associated virus serotype 5
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 13 - DNA, 142 bp, unidentified
  source 1..142: mol_type = genomic DNA; note = Adeno-associated virus serotype 6
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 14 - DNA, 143 bp, unidentified; identical to SEQ ID NO: 8
  source 1..143: mol_type = genomic DNA; note = Adeno-associated virus serotype rh74

SEQ ID NO: 15 - protein, 738 aa, unidentified
  source 1..738: note = Adeno-associated virus serotype rh74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 16 - protein, 736 aa, unidentified
  source 1..736: note = Adeno-associated virus serotype 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - protein, 735 aa, unidentified [sequence omitted]

SEQ ID NO: 18 - protein, 736 aa, unidentified
  source 1..736: note = Adeno-associated virus serotype 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 19 - protein, 734 aa, unidentified
  source 1..734: note = Adeno-associated virus serotype 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 20 - protein, 724 aa, unidentified
  source 1..724: note = Adeno-associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 736 aa, unidentified
  source 1..736: note = Adeno-associated virus serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - protein, 737 aa, unidentified
  source 1..737: note = Adeno-associated virus serotype 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 23 - protein, 738 aa, unidentified
  source 1..738: note = Adeno-associated virus serotype 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - protein, 736 aa, unidentified
  source 1..736: note = Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 738 aa, unidentified
  source 1..738: note = Adeno-associated virus serotype 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 26 - protein, 733 aa, unidentified
  source 1..733: note = Adeno-associated virus serotype 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 27 - protein, 742 aa, unidentified
  source 1..742: note = Adeno-associated virus serotype 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 28 - protein, 738 aa, unidentified; identical to SEQ ID NO: 25
  source 1..738: note = Adeno-associated virus serotype rh10

SEQ ID NO: 29 - DNA, 2217 bp, unidentified
  source 1..2217: mol_type = genomic DNA; note = Adeno-associated virus serotype rh74
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgccgatcctccgaccaccttcaatcaggccaagctggcttctttcatcacgcagtacagtaccggtcaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 30 - DNA, 145 bp, unidentified
  source 1..145: mol_type = genomic DNA; note = Adeno-associated virus serotype 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 31 - DNA, 136 bp, synthetic construct
  source 1..136: mol_type = other DNA
tcgtcgacgagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 32 - DNA, 146 bp, synthetic construct
  source 1..146: mol_type = other DNA
gataaaaatctcgtcgacgagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 33 - DNA, 10 bp, synthetic construct
  source 1..10: mol_type = other DNA
gatttttatc

SEQ ID NO: 34 - DNA, 10 bp, synthetic construct
  source 1..10: mol_type = other DNA
gattttaatc

SEQ ID NO: 35 - DNA, 10 bp, synthetic construct
  source 1..10: mol_type = other DNA
gatttaaatc

SEQ ID NO: 36 - DNA, 19 bp, synthetic construct
  source 1..19: mol_type = other DNA
tcgtcgacggatttttatc

SEQ ID NO: 37 - DNA, 19 bp, synthetic construct
  source 1..19: mol_type = other DNA
agcagctgcctaaaaatag

(19) United States
(12) Patent Application Publication
(10) Pub. No.: US 2025/0121094 A1
(43) Pub. Date: Apr. 17, 2025

(54) AAV PARTICLES WITH MODIFIED INVERTED TERMINAL REPEATS FOR ENHANCED GENE EXPRESSION IN MUSCLE

(71) Applicant: University of Florida Research Foundation, Incorporated, Gainesville, FL (US)

(72) Inventors: Arun Srivastava, Gainesville, FL (US); Keyun Qing, Gainesville, FL (US); Jakob Shoti, Gainesville, FL (US)

(73) Assignee: University of Florida Research Foundation, Incorporated, Gainesville, FL (US)

(21) Appl. No.: 18/692,493

(22) PCT Filed: Sep. 16, 2022
(86) PCT No.: PCT/US2022/076591

§ 371 Date: Mar. 15, 2024

Related U.S. Application Data
(60) Provisional application No. 63/244,759, filed on Sep. 16, 2021.

(51) Int. Cl. A61K 48/00; C12N 15/86

(52) U.S. Cl. CPC A61K 48/0058; C12N 15/86; C12N 2750/14143; C12N 2750/14145

(57) ABSTRACT

Provided herein are AAV inverted terminal repeats (ITRs) comprising modifications that are useful for improved expression of a transgene to muscle cells/tissue. ITRs as described herein comprise a myoblast determination protein (MyoD) binding site and/or myocyte enhancer factor (MEF) binding site. to improve transgene expression in muscle cells/tissue. Provided herein are also AAV particles comprising modified ITRs. and method of making and using them.

### RELATED APPLICATIONS

This application claims priority under 35 U.S.C. § 119(e) to U.S. Provisional Application Ser. No. 63/244,759, entitled “AAV PARTICLES WITH MODIFIED INVERTED TERMINAL REPEATS FOR ENHANCED GENE EXPRESSION IN MUSCLE”, filed on Sep. 16, 2021, the contents of which are incorporated herein by reference in their entirety.

### REFERENCE TO AN ELECTRONIC SEQUENCE LISTING

The contents of the electronic sequence listing (U120270081WO00-SEQ-COB.xml; Size: 51,572 bytes; and Date of Creation: Sep. 13, 2022) are herein incorporated by reference in their entirety.

### BACKGROUND

Gene therapy has the potential to treat subject suffering from or are at risk of suffering from genetic disease. Improved AAV vectors for carrying genetic payload would be beneficial to the development of gene therapies, e.g., for certain diseases that affect muscle tissue and/or function. Muscle diseases, such as muscular dystrophies, can result from numerous conditions including, for example, congenital or acquired somatic mutations, injury, and exposure to hazardous compounds. In some cases, muscle diseases result in life-threatening complications or lead to serious symptoms and/or death. Although numerous factors have been implicated in regulating muscle diseases, including muscular dystrophies, effective treatments remain limited.

### SUMMARY

The present disclosure is based at least in part on the realization many muscle-related genes require myoblast determination protein (MyoD) and/or myocyte enhancer factor (MEF) to activate transcription. As a strategy to improve the amount of transgene delivery to muscle cells/tissue, the present disclosure provides AAV nucleic acid vectors comprising a 5′ inverted terminal repeat (ITR) comprising a MyoD and/or MEF binding site; and particles comprising them.

In some aspects, provided herein is adeno-associated virus (AAV) particle comprising a nucleic acid vector, wherein the nucleic acid vector comprises a 5′ inverted terminal repeat (ITR) comprising a myoblast determination protein (MyoD) binding site and/or myocyte enhancer factor (MEF) binding site.

In some embodiments, a 5′ ITR comprises a MyoD binding site. In some embodiments, a MyoD binding site comprises, consists of, or consists essentially of the nucleic acid sequence 5′-AGCAGCTGCT-3′ (SEQ ID NO: 1). In some embodiments, a MyoD binding site comprises, consists of, or consists essentially of the nucleic acid sequence 5′-TCGTCGACG-3′. In some embodiments, a MyoD binding site comprises, consists of, or consists essentially of the nucleic acid sequence 5′-AGCAGCTGC-3′. In some embodiments, a 5′ ITR comprises a MEF binding site. In some embodiments, a MEF binding site comprises, consists of, or consists essentially of the nucleic acid sequence 5′-CTAAAAATAG-3′ (SEQ ID NO: 4). In some embodiments, a MEF binding site comprises, consists of, or consists essentially of the nucleic acid sequence 5′-GATTTTTATC-3′ (SEQ ID NO: 33). In some embodiments, a 5′ ITR comprises a MyoD binding site and a MEF binding site. In some embodiments, a MEF binding site is downstream of a MyoD binding site in an ITR. In some embodiments, a MyoD and/or MEF binding sites are comprised downstream from the terminal resolution site of the ITR.

In some embodiments, a nucleic acid vector comprising any one of the ITRs as described herein further comprises a transgene. In some embodiments, a nucleic acid vector comprising any one of the ITRs as described herein further comprises a promoter, e.g., a muscle-specific promoter. In some embodiments, an AAV particle is an AAVrh74 particle. In some embodiments, a nucleic acid vector is of serotype 2, e.g., comprising the sequence of SEQ ID NO: 9.

In some aspects, provided herein is an AAV particle comprising any one of the nucleic acid vectors described herein. In some embodiments, the transduction efficiency of an AAV particle as described herein is at least 2 times higher than the transduction efficiency of an AAV particle comprising a 5′ ITR lacking MyoD and/or MEF binding sites.

In some aspects, provided herein is a composition comprising any one of the AAV particles described herein. In some embodiments, a composition comprising AAV particles comprises a pharmaceutically acceptable carrier.

Provided herein, in some aspects, is a method comprising delivering to a cell or administering to a subject any one of the particles, or composition comprising any one of the particles, described herein. In some embodiments, a subject is human.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows an example of a wild-type ITR of AAV2 with secondary structure in Flip configuration. The boxed motif corresponds to the Rep-binding element (RBE) to which AAV Rep78 and Rep68 proteins bind. The RBE consists of a tetranucleotide repeat with the consensus sequence 5′-GNGC-3′. The ATP-dependent DNA helicase activities of Rep78 and Rep68 remodel the A-A′ region generating a stem-loop that locates at the summit the terminal resolution site (trs) in a single-stranded form. The strand-and site-specific endonuclease catalytic domain of Rep78 and Rep68 introduces a nick at the trs. The RBE′ stabilizes the association between the two largest Rep proteins and the ITR.

FIG. 2A exemplifies a 5′ ITR comprising a MyoD binding site.

FIG. 2B exemplifies a 5′ ITR comprising a MyoD and MEF binding site.

### DETAILED DESCRIPTION

Provided herein are compositions and methods useful for delivery of a transgene to muscle cells or tissue. In some embodiments of the strategy provided here, a myoblast determination protein (MyoD) binding site and/or myocyte enhancer factor (MEF) binding site is comprised in a 5′ ITR of an AAV particle. The AAV capsid proteins, particles comprising them, compositions comprising the particles can be used in a variety of applications including but not limited to methods of treating a subject suffering from or at risk of suffering from a disease or disorder (e.g., a muscular dystrophy) by delivering one or more genes of interest to a particular tissue or organ.

### ITRs Comprising MyOD and/or MEF Binding Sites

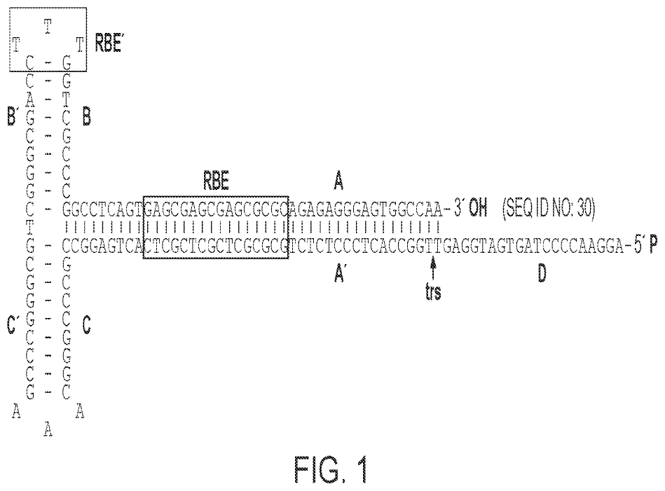

Provided herein are AAV inverted terminal repeats modified to improve transgene expression in muscle cells or muscle tissue. In some embodiments, an ITR as provided herein is a 5′ ITR, i.e. an ITR that is 5′ from a transgene on a nucleic acid vector that is encapsidated by an AAV capsid. FIG. 1 provides an example of a wild-type ITR with secondary structure. An ITR serves as an origin of replication and is comprised of two arm palindromes (B-B′ and C-C′) embedded in a larger stem palindrome (A-A′). An AAV ITR can be in flip or flop configurations. See e.g., Human Gene Therapy Methods 23(2):128-36. In some embodiments, and ITR has the B-B′ and the C-C′ palindrome closest to the 3′ end. In wild-type ITRs, the D sequence is present only once at each end of the genome thus remaining single-stranded.

In some embodiments, an ITR (e.g., a 5′ ITR) as provided herein comprises a myoblast determination protein (MyoD) binding site and/or myocyte enhancer factor (MEF) binding site. In some embodiments, an ITR (e.g., a 5′ ITR) comprises a MyoD binding site. MyoD is a transcriptional activator that promotes transcription of muscle-specific target genes and plays a role in muscle differentiation. In some embodiments, MyoD is human MyoD. In some embodiments, MyoD is a non-human MyoD, such as murine MyoD. In some embodiments, a MyoD binding site comprises the nucleic acid sequence 5′-AGCAGCTGCT-3′ (SEQ ID NO: 1), or a sequence that is at least 50% (e.g., at least 50%, at least 60%, at least 70%, at least 80%, at least 85%, at least 90%, at least 95%, at least 97%, at least 98%, at least 99%) identical to SEQ ID NO: 1 and can bind MyoD. In some embodiments, a MyoD binding site comprises the nucleic acid sequence 5′-AGCAGCTGC-3′, or a sequence that is at least 50% (e.g., at least 50%, at least 60%, at least 70%, at least 80%, at least 85%, at least 90%, at least 95%, at least 97%, at least 98%, at least 99%) identical to the nucleic acid sequence 5′-AGCAGCTGC-3′ and can bind MyoD. In some embodiments, a MyoD binding site comprises the nucleic acid sequence 5′-TCGTCGACG-3′, or a sequence that is at least 50% (e.g., at least 50%, at least 60%, at least 70%, at least 80%, at least 85%, at least 90%, at least 95%, at least 97%, at least 98%, at least 99%) identical to the nucleic acid sequence 5′-TCGTCGACG-3′ and can bind MyoD. In some embodiments, an ITR as provided herein comprises a MyoD binding site that has a sequence comprising 1, 2, 3, 4, 5, 6, or 7 nucleotide substitutions in SEQ ID NO: 1. For example a MyoD binding site may have a sequence 5′-AGCCGCTGCT-3′ (SEQ ID NO: 2) or 5′-ACAAGCTGCT-3′ (SEQ ID NO: 3). In some embodiments, an ITR as provided herein comprises a MyoD binding site that has a sequence comprising 1, 2, 3, 4, 5, 6, or 7 nucleotide substitutions in the nucleic acid sequence 5′-AGCAGCTGC-3′. For example, a MyoD binding site may have a sequence 5′-AGCCGCTGC-3′ or 5′-ACAAGCTGC-3′. In some embodiments, an ITR as provided herein comprises a MyoD binding site that has a sequence comprising 1, 2, 3, 4, 5, 6, or 7 nucleotide substitutions in the nucleic acid sequence 5′-TCGTCGACG-3′. For example, a MyoD binding site may have a sequence 5′-TCGTCGCCG-3′ or 5′-TCGTCGAAC-3′. In some embodiments, a MyoD binding site has a sequence that a binding capacity to MyoD that is the same as the binding capacity of a MyoD binding site with a sequence of SEQ ID NO: 1. In some embodiments, the binding capacity of a MyoD binding site as comprised in any one of the ITRs provided herein is 10-1000% (10-1000, 10-20, 10-50, 20-100, 40-100, 50-100, 60-100, 70-100,80-100, 90-100, 50-150, 100-150, 100-200, 100-500, or 500-1000%) of the binding capacity of a MyoD binding site having the sequence of SEQ ID NO: 1, 2, or 3. In some embodiments, the binding capacity of a MyoD binding site as comprised in any one of the ITRs provided herein is 10-1000% (10-1000, 10-20, 10-50, 20-100, 40-100, 50-100, 60-100, 70-100,80-100, 90-100, 50-150, 100-150, 100-200, 100-500, or 500-1000%) of the binding capacity of a MyoD binding site having the sequence of 5′-AGCAGCTGC-3′, 5′-AGCCGCTGC-3′ or 5′-ACAAGCTGC-3′, 5′-TCGTCGACG-3′, 5′-TCGTCGCCG-3′ or 5′-TCGTCGAAC-3′.

In some embodiments, an ITR (e.g., a 5′ ITR) comprises a myocyte enhancer factor (MEF) binding site. Myocyte enhancer factor 2 (MEF2) family proteins are key transcription factors controlling gene expression in myocytes. In some embodiments, MEF is human MEF. In some embodiments, MEF is a non-human MEF, such as murine MEF. In some embodiments, a MEF binding site comprises the nucleic acid sequence 5′-CTAAAAATAG-3′ (SEQ ID NO: 4), or a sequence that is at least 50% (e.g., at least 50%, at least 60%, at least 70%, at least 80%, at least 85%, at least 90%, at least 95%, at least 97%, at least 98%, at least 99%) identical to SEQ ID NO: 4 and can bind MEF. In some embodiments, a MEF binding site comprises the nucleic acid sequence 5′-GATTTTTATC-3′ (SEQ ID NO: 33), or a sequence that is at least 50% (e.g., at least 50%, at least 60%, at least 70%, at least 80%, at least 85%, at least 90%, at least 95%, at least 97%, at least 98%, at least 99%) identical to SEQ ID NO: 33 and can bind MEF. In some embodiments, an ITR as provided herein comprises a MEF binding site that has a sequence comprising 1, 2, 3, 4, 5, 6, or 7 nucleotide substitutions in SEQ ID NO: 4. For example a MEF binding site may have a sequence 5′-CTAAAATTAG-3′ (SEQ ID NO: 5) or 5′-CTAAATTTAG-3′ (SEQ ID NO: 6). In some embodiments, an ITR as provided herein comprises a MEF binding site that has a sequence comprising 1, 2, 3, 4, 5, 6, or 7 nucleotide substitutions in the sequence 5′-GATTTTTATC-3′ (SEQ ID NO: 33). For example, a MEF binding site may have a sequence 5′-GATTTTAATC-3′ (SEQ ID NO: 34) or 5′-GATTTAAATC-3′ (SEQ ID NO: 35). In some embodiments, a MEF binding site has a sequence that has a binding capacity to MEF that is the same as the binding capacity of a MEF binding site with a sequence of SEQ ID NO: 1. In some embodiments, the binding capacity of a MEF binding site as comprised in any one of the ITRs provided herein is 10-1000% (10-1000, 10-20, 10-50, 20-100, 40-100, 50-100, 60-100, 70-100, 80-100, 90-100, 50-150, 100-150, 100-200, 100-500, or 500-1000%) of the binding capacity of a MEF binding site having the sequence of a MEF binding site disclosed herein (e.g., having the sequence of SEQ ID NO: 4, 5, 6, 33, 34, or 35).

In some embodiments, an ITR as provided herein comprises both a MyoD binding site and a MEF binding site.

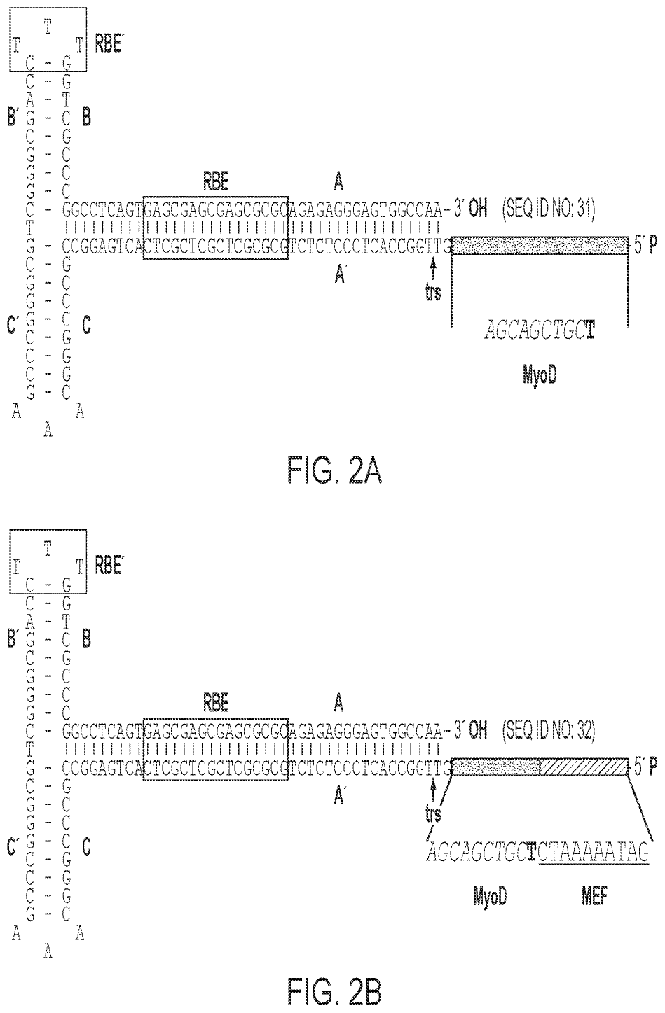

In some embodiments, an ITR comprises a MyoD binding site and/or a MEF binding site downstream of or 3′ to the terminal resolution site (trs) of the ITR. In some embodiments, a MyoD binding site and/or a MEF binding site is immediately after the trs. In some embodiments, a MyoD binding site and/or a MEF binding site is 1, 2, 3, 4, 5, 6, 7, 8, 9, or 10 nucleotides downstream from the trs of the ITR. In some embodiments, a MyoD binding site and/or a MEF binding site in the ITR replaces part of or the entire D sequence of the ITR. See e.g., FIGS. 2A and 2B. In some embodiments, a D sequence of an ITR is about 20 (e.g., about 15, 16, 17, 18, 19, 20, 21, 22, 23, 24, or 25) nucleotides located downstream from or 3′ to the trs of the ITR. In some embodiments, a D sequence of an ITR corresponds to the sequence CTCCATCACTAGGGGTTCCT (SEQ ID NO: 7) of wild-type AAV2, or a fragment thereof (e.g., 1, 2, 3, 4, 5, 6, 7, 8, 9, 10, 11, 12, 13, 14, 15, 16, 17, 18, or 19 nucleotides long). In some embodiments, an ITR as provided herein does not comprise a D sequence. SEQ ID NOs: 8-14 provide ITR sequences of nucleic acid vectors of AAV particles, in which nucleic acid vectors, ITRs can be modified to introduce a MyoD and/or MEF binding site.

In some embodiments, an ITR comprises a MyoD binding site and/or a MEF binding site upstream of or 5′ to the terminal resolution site (trs) of the ITR. In some embodiments, a MyoD binding site and/or a MEF binding site is immediately adjacent to the trs. In some embodiments, a MyoD binding site and/or a MEF binding site is 1, 2, 3, 4, 5, 6, 7, 8, 9, or 10 nucleotides upstream from the trs of the ITR. In some embodiments, a MyoD binding site and/or a MEF binding site in the ITR replaces part of or the entire D sequence of the ITR. See e.g., FIGS. 2A and 2B. In some embodiments, a D sequence of an ITR is about 20 (e.g., about 15, 16, 17, 18, 19, 20, 21, 22, 23, 24, or 25) nucleotides located upstream from or 5′ to the trs of the ITR. In some embodiments, a D sequence of an ITR corresponds to the sequence CTCCATCACTAGGGGTTCCT (SEQ ID NO: 7) of wild-type AAV2, or a fragment thereof (e.g., 1, 2, 3, 4, 5, 6, 7, 8, 9, 10, 11, 12, 13, 14, 15, 16, 17, 18, or 19 nucleotides long). In some embodiments, an ITR as provided herein does not comprise a D sequence. SEQ ID NOs: 8-14 provide ITR sequences of nucleic acid vectors of AAV particles, in which nucleic acid vectors, ITRs can be modified to introduce a MyoD and/or MEF binding site.

In some embodiments, an ITR comprises a sequence arrangement of 5′-[MyoD binding site]-[MEF binding site]-3′. In some embodiments, an ITR comprises a sequence arrangement of 5′-[MEF binding site]-[MyoD binding site]-3′. For example, in some embodiments, an ITR comprises a sequence 5′-TCGTCGACG-GATTTTTATC-3′ (SEQ ID NO: 36) or 5′-AGCAGCTGC-CTAAAAATAG-3′ (SEQ ID NO: 37).

Example of wild-type AAV1 left ITR:
(SEQ ID NO: 8)
TTGCCCACTCCCTCTCTGCGCGCTCGCTCGCTCGGTGGGGCCTGCGGACC
AAAGGTCCGCAGACGGCAGAGGTCTCCTCTGCCGGCCCCACCGAGCGAGC
GAGCGCGCAGAGAGGGAGTGGGCAACTCCATCACTAGGGGTAA
Example of wild-type AAV2 left ITR:
(SEQ ID NO: 9)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT
Example of wild-type AAV3 left ITR:
(SEQ ID NO: 10)
TTGGCCACTCCCTCTATGCGCACTCGCTCGCTCGGTGGGGCCTGGCGACC
AAAGGTCGCCAGACGGACGTGCTTTGCACGTCCGGCCCCACCGAGCGAGC
GAGTGCGCATAGAGGGAGTGGCCAACTCCATCACTAGAGGTATGGC
Example of wild-type AAV4 left ITR:
(SEQ ID NO: 11)
TTGGCCACTCCCTCTATGCGCGCTCGCTCACTCACTCGGCCCTGGAGACC
AAAGGTCTCCAGACTGCCGGCCTCTGGCCGGCAGGGCCGAGTGAGTGAGC
GAGCGCGCATAGAGGGAGTGGCCAACTCCATCATCTAGGTTTGCCC
Example of wild-type AAV5 left ITR:
(SEQ ID NO: 12)
CTCTCCCCCCTGTCGCGTTCGCTCGCTCGCTGGCTCGTTTGGGGGGGTGG
CAGCTCAAAGAGCTGCCAGACGACGGCCCTCTGGCCGTCGCCCCCCCAAA
CGAGCCAGCGAGCGAGCGAACGCGACAGGGGGGAGAGTGCCACACTCTCA
AGCAAGGGGGTTTTGTA
Example of wild-type AAV6 left ITR:
(SEQ ID NO: 13)
TTGCCCACTCCCTCTATGCGCGCTCGCTCGCTCGGTGGGGCCTGCGGACC
AAAGGTCCGCAGACGGCAGAGCTCTGCTCTGCCGGCCCCACCGAGCGAG
CGAGCGCGCATAGAGGGAGTGGGCAACTCCATCACTAGGGGTA
Example of wild-type AAVrh74 left ITR:
(SEQ ID NO: 14)
TTGCCCACTCCCTCTCTGCGCGCTCGCTCGCTCGGTGGGGCCTGCGGACC
AAAGGTCCGCAGACGGCAGAGGTCTCCTCTGCCGGCCCCACCGAGCGAGC
GAGCGCGCAGAGAGGGAGTGGGCAACTCCATCACTAGGGGTAA

In ITRs comprising both a MyoD binding site and a MEF binding site, the MyoD binding site can be upstream from (or 5′ relative to) or downstream from (or 3′ relative to) the MEF binding site. In some embodiments, there are no nucleotides between the MyoD and MEF binding sites. In some embodiments, there are 1, 2, 3, 4, 5, 6, 7,9, or 10 nucleotides between the MyoD and MEF binding sites. An example of an ITR comprising both a MyoD and MEF binding sites is shown in FIG. 2B.

In some embodiments, an ITR comprises more than one MyoD binding site, or more than one MEF binding site. For example an ITR may comprise two copies of a MyoD binding site, each comprising the sequence of SEQ ID NO: 1. Any number or arrangement of MyoD and/or MEF binding sites are contemplated herein. For example, an ITR may comprise the configuration: MyoD binding site—MEF binding site—MyoD binding site, MyoD binding site—MyoD binding site—MEF binding site.

### AAV Nucleic Acid Vectors Comprising a 5′ ITR

Provided herein are nucleic acid vectors that comprise the ITRs are described herein. In some embodiments, a nucleic acid vector as provided herein is encapsidated in an AAV particle by capsid protein.

In some embodiments, an AAV nucleic acid vector comprises a transgene. In some embodiments, a transgene is located between two ITRs, a 5′ ITR and a 3′ ITR. In some embodiments, a transgene encodes a therapeutic molecule. A therapeutic molecule may be an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, an anticoagulant, a nuclease, guide RNA or other nucleic acid or protein for gene editing, or any functional portion of any of these molecules. In some embodiments, a therapeutic molecule, such as a therapeutic protein, is one that affects muscle function. For example, a therapeutic molecule may be a protein that is implicated in a muscular dystrophy. Non-limiting examples of proteins implicated in a muscular dystrophy are dystrophin, myotilin, lamin, caveolin, caplain-3, dysferlin, a sarcoglycan, AUF1, TCAP, TRIM32, FKRP, titin, acetylflucosamine epimerase, Desmin, LARGE, fukutin, an integrin, salenoprotein, a collagen, and plectin. Lovering et al. (Phys Ther. 2005 December; 85(12): 1372-1388), provides examples of muscular dystrophies and implicated proteins that can be targeted for therapy.

In some embodiments, an AAV nucleic acid vector comprises one or more regulatory elements that are operably linked to a transgene. In some embodiments, a regulatory element is located between two ITRs, a 5′ ITR and a 3′ ITR. In some embodiments, a regulatory element is located upstream of or 5′ relative to a transgene. In some embodiments, a regulatory element is located downstream of or 3′ relative to the 5′ ITRs as described herein. In some embodiments, a regulatory element is located upstream of or 5′ relative to a transgene and downstream of or 3′ relative to a 5′ ITR.

A regulatory element refers to a nucleotide fragment or structural component of a nucleic acid which is involved in the regulation of expression of components of the nucleic acid vector (e.g., a transgene comprised in the nucleic acid vector). Regulatory elements include, but are not limited to, promoters, enhancers, silencers, insulators, response elements, initiation sites, termination signals, and ribosome binding sites.

Promoters include constitutive promoters, inducible promoters, tissue-specific promoters, cell type-specific promoters, and synthetic promoters. For example, a nucleic acid vector disclosed herein may include viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters or other inducible regulatory elements may also be used to achieve desired expression levels of a gene of interest (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters or other tissue-specific regulatory elements are also contemplated herein. Non-limiting examples of such promoters that may be used include muscle-specific promoters. An example of a muscle-specific promoter is MHCK7.

Synthetic promoters are also contemplated herein. A synthetic promoter may comprise, for example, regions of known promoters, regulatory elements, transcription factor binding sites, enhancer elements, repressor elements, and the like.

In some embodiments, a transgene encodes a detectable molecule. A detectable molecule is one that can be detected in a sample of tissue or an organ or in a subject body by some imaging method. In some embodiments, a detectable molecule is a fluorescent, bioluminescent, radiolabeled, or enzymatic protein or functional peptide or functional polypeptide thereof.

Additional features of AAV particles, nucleic acid vectors encapsidated in them, and capsid proteins are described in U.S. Patent Publication No. 2017/0356009, the contents of which are incorporated herein by reference in their entirety.

### AAV Particles

Provided herein are AAV particles that comprise any of the AAV nucleic acid vectors disclosed herein. AAV particles may be of any serotype (e.g., or serotype 1, serotype 2, serotype 3, serotype 4, serotype 5, serotype 6, serotype 7, serotype 8, serotype 9, serotype 10, serotype rh10, serotype 11, serotype 12, serotype 13, or serotype rh74). In some embodiments, an AAV particle a provided herein comprises a capsid of a first serotype and a nucleic acid vector of a second serotype. In some embodiments, the first and second serotypes are the same. For example, an AAV particle as provided herein may comprise a capsid of serotype rh74 that encapsidates nucleic acid vector of serotype rh74. In some embodiments, the first and second serotypes are different. For example, an AAV particle as provided herein may comprise a capsid of serotype rh74 that encapsidates nucleic acid vector of serotype 2. SEQ ID NOs. 15-28 provide examples of amino acid sequences of AAV capsid proteins of different serotypes.

```
Example of an amino acid sequence of AAVrh74 capsid protein:
(SEQ ID NO: 15)
    1 MAADGYLPDW LEDNLSEGIR EWWDLKPGAP KPKANQQKQD NGRGLVLPGY
  51 KYLGPENGLD KGEPVNAADA AALEHDKAYD QQLQAGDNPY LRYNHADAEF
101 QERLQEDTSF GGNLGRAVFQ AKKRVLEPLG LVESPVKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGTSGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVEMIPQYGY LTLNNGSQAV GRSSFYCLEY
401 FPSQMLRIGN NFEFSYNFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTENQAKLA SFITQYSTGQ VSVEIEWELQ KENSKRWNPE
701 IQYTSNYYKS TNVDFAVNTE GTYSEPRPIG TRYLTRNL
Example of an amino acid sequence of wild-type AAV1 capsid protein
(SEQ ID NO: 16)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEQSP
151 QEPDSSSGIG KTGQQPAKKR LNFGQTGDSE SVPDPQPLGE PPATPAAVGP
201 TTMASGGGAP MADNNEGADG VGNASGNWHC DSTWLGDRVI TTSTRTWALP
251 TYNNHLYKQI SSASTGASND NHYFGYSTPW GYFDFNRFHC HFSPRDWQRL
301 INNNWGFRPK RLNFKLFNIQ VKEVTINDGV TTIANNLIST VQVFSDSEYQ
351 LPYVLGSAHQ GCLPPFPADV FMIPQYGYLT LNNGSQAVGR SSFYCLEYFP
401 SQMLRTGNNF TFSYTFEEVP FHSSYAHSQS LDRLMNPLID QYLYYLNRTQ
451 NQSGSAQNKD LLFSRGSPAG MSVQPKNWLP GPCYRQQRVS KTKTDNNNSN
501 FTWTGASKYN LNGRESIINP GTAMASHKDD EDKFFPMSGV MIFGKESAGA
551 SNTALDNVMI TDEEEIKATN PVATERFGTV AVNFQSSSTD PATGDVHAMG
601 ALPGMVWQDR DVYLQGPIWA KIPHTDGHFH PSPLMGGFGL KNPPPQILIK
651 NTPVPANPPA EFSATKFASF ITQYSTGQVS VEIEWELQKE NSKRWNPEVQ
701 YTSNYAKSAN VDFTVDNNGL YTEPRPIGTR YLTRPL
Example of an amino acid sequence of wild-type AAV2 capsid protein
(SEQ ID NO: 17)
    1 MAADGYLPDW LEDTLSEGIR QWWKLKPGPP PPKPAERHKD DSRGLVLPGY
  51 KYLGPFNGLD KGEPVNEADA AALEHDKAYD RQLDSGDNPY LKYNHADAEF
101 QERLKEDTSF GGNLGRAVFQ AKKRVLEPLG LVEEPVKTAP GKKRPVEHSP
151 VEPDSSSGTG KAGQQPARKR LNFGQTGDAD SVPDPQPLGQ PPAAPSGLGT
201 NTMATGSGAP MADNNEGADG VGNSSGNWHC DSTWMGDRVI TTSTRTWALP
251 TYNNHLYKQI SSQSGASNDN HYFGYSTPWG YFDFNRFHCH FSPRDWQRLI
301 NNNWGFRPKR LNFKLFNIQV KEVTQNDGTT TIANNLTSTV QVFTDSEYQL
351 PYVLGSAHQG CLPPFPADVF MVPQYGYLTL NNGSQAVGRS SFYCLEYFPS
401 QMLRTGNNFT FSYTFEDVPF HSSYAHSQSL DRLMNPLIDQ YLYYLSRINT
451 PSGTTTQSRL QFSQAGASDI RDQSRNWLPG PCYRQQRVSK TSADNNNSEY
501 SWTGATKYHL NGRDSLVNPG PAMASHKDDE EKFFPQSGVL IFGKQGSEKT
551 NVDIEKVMIT DEEEIRTINP VATEQYGSVS TNLQRGNRQA ATADVNTQGV
601 LPGMVWQDRD VYLQGPIWAK IPHTDGHFHP SPLMGGFGLK HPPPQILIKN
651 TPVPANPSTT FSAAKFASFI TQYSTGQVSV EIEWELQKEN SKRWNPEIQY
701 TSNYNKSVNV DFTVDINGVY SEPRPIGTRY LTRNL
Example of an amino acid sequence of wild-type AAV3 capsid protein
(SEQ ID NO: 18)
    1 MAADGYLPDW LEDNLSEGIR EWWALKPGVP QPKANQQHQD NRRGLVLPGY
  51 KYLGPGNGLD KGEPVNEADA AALEHDKAYD QQLKAGDNPY LKYNHADAEF
101 QERLQEDTSF GGNLGRAVFQ AKKRILEPLG LVEEAAKTAP GKKGAVDQSP
151 QEPDSSSGVG KSGKQPARKR LNFGQTGDSE SVPDPQPLGE PPAAPTSLGS
201 NTMASGGGAP MADNNEGADG VGNSSGNWHC DSQWLGDRVI TTSTRTWALP
251 TYNNHLYKQI SSQSGASNDN HYFGYSTPWG YFDFNRFHCH FSPRDWQRLI
301 NNNWGFRPKK LSFKLFNIQV RGVTQNDGTT TIANNLTSTV QVFTDSEYQL
351 PYVLGSAHQG CLPPFPADVF MVPQYGYLTL NNGSQAVGRS SFYCLEYFPS
401 QMLRTGNNFQ FSYTFEDVPF HSSYAHSQSL DRLMNPLIDQ YLYYLNRTQG
451 TTSGTTNQSR LLFSQAGPQS MSLQARNWLP GPCYRQQRLS KTANDNNNSN
501 FPWTAASKYH LNGRDSLVNP GPAMASHKDD EEKFFPMHGN LIFGKEGTTA
551 SNAELDNVMI TDEEEIRTTN PVATEQYGTV ANNLQSSNTA PTTGTVNHQG
601 ALPGMVWQDR DVYLQGPIWA KIPHTDGHFH PSPLMGGFGL KHPPPQIMIK
651 NTPVPANPPT TFSPAKFASF ITQYSTGQVS VEIEWELQKE NSKRWNPEIQ
701 YTSNYNKSVN VDFTVDINGV YSEPRPIGTR YLTRNL
Example of an amino acid sequence of wild-type AAV4 capsid protein
(SEQ ID NO: 19)
    1 MTDGYLPDWL EDNLSEGVRE WWALQPGAPK PKANQQHQDN ARGLVLPGYK
  51 YLGPGNGLDK GEPVNAADAA ALEHDKAYDQ QLKAGDNPYL KYNHADAEFQ
101 QRLQGDTSFG GNLGRAVFQA KKRVLEPLGL VEQAGETAPG KKRPLIESPQ
151 QPDSSTGIGK KGKQPAKKKL VFEDETGAGD GPPEGSTSGA MSDDSEMRAA
201 AGGAAVEGGQ GADGVGNASG DWHCDSTWSE GHVTTTSTRT WVLPTYNNHL
251 YKRLGESLQS NTYNGFSTPW GYFDFNRFHC HFSPRDWQRL INNNWGMRPK
301 AMRVKIFNIQ VKEVTTSNGE TTVANNLIST VQIFADSSYE LPYVMDAGQE
351 GSLPPFPNDV FMVPQYGYCG LVTGNTSQQQ TDRNAFYCLE YFPSQMLRTG
401 NNFEITYSFE KVPFHSMYAH SQSLDRLMNP LIDQYLWGLQ STTTGTTLNA
451 GTATTNFTKL RPTNFSNFKK NWLPGPSIKQ QGFSKTANQN YKIPATGSDS
501 LIKYETHSTL DGRWSALTPG PPMATAGPAD SKFSNSQLIF AGPKQNGNTA
551 TVPGTLIFTS EEELAATNAT DTDMWGNLPG GDQSNSNLPT VDRLTALGAV
601 PGMVWQNRDI YYQGPIWAKI PHTDGHFHPS PLIGGFGLKH PPPQIFIKNT
651 PVPANPATTF SSTPVNSFIT QYSTGQVSVQ IDWEIQKERS KRWNPEVQFT
701 SNYGQQNSLL WAPDAAGKYT EPRAIGTRYL THHL
Example of an amino acid sequence of wild-type AAV5 capsid protein
(SEQ ID NO: 20)
    1 MSFVDHPPDW LEEVGEGLRE FLGLEAGPPK PKPNQQHQDQ ARGLVLPGYN
  51 YLGPGNGLDR GEPVNRADEV AREHDISYNE QLEAGDNPYL KYNHADAEFQ
101 EKLADDTSFG GNLGKAVFQA KKRVLEPFGL VEEGAKTAPT GKRIDDHFPK
151 RKKARTEEDS KPSTSSDAEA GPSGSQQLQI PAQPASSLGA DTMSAGGGGP
201 LGDNNQGADG VGNASGDWHC DSTWMGDRVV TKSTRTWVLP SYNNHQYREI
251 KSGSVDGSNA NAYFGYSTPW GYFDFNRFHS HWSPRDWQRL INNYWGFRPR
301 SLRVKIFNIQ VKEVTVQDST TTIANNLIST VQVFTDDDYQ LPYVVGNGTE
351 GCLPAFPPQV FTLPQYGYAT LNRDNTENPT ERSSFFCLEY FPSKMLRTGN
401 NFEFTYNFEE VPFHSSFAPS QNLFKLANPL VDQYLYRFVS INNTGGVQFN
451 KNLAGRYANT YKNWFPGPMG RTQGWNLGSG VNRASVSAFA TTNRMELEGA
501 SYQVPPQPNG MTNNLQGSNT YALENTMIFN SQPANPGTTA TYLEGNMLIT
551 SESETQPVNR VAYNVGGQMA TNNQSSTTAP ATGTYNLQEI VPGSVWMERD
601 VYLQGPIWAK IPETGAHFHP SPAMGGFGLK HPPPMMLIKN TPVPGNITSF
651 SDVPVSSFIT QYSTGQVTVE MEWELKKENS KRWNPEIQYT NNYNDPQFVD
701 FAPDSTGEYR TTRPIGTRYL TRPL
Example of an amino acid sequence of wild-type AAV6 capsid protein
(SEQ ID NO: 21)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPFG LVEEGAKTAP GKKRPVEQSP
151 QEPDSSSGIG KTGQQPAKKR LNFGQTGDSE SVPDPQPLGE PPATPAAVGP
201 TTMASGGGAP MADNNEGADG VGNASGNWHC DSTWLGDRVI TTSTRTWALP
251 TYNNHLYKQI SSASTGASND NHYFGYSTPW GYFDFNRFHC HFSPRDWQRL
301 INNNWGFRPK RLNFKLFNIQ VKEVTINDGV TTIANNLIST VQVFSDSEYQ
351 LPYVLGSAHQ GCLPPFPADV FMIPQYGYLT LNNGSQAVGR SSFYCLEYFP
401 SQMLRTGNNF TFSYTFEDVP FHSSYAHSQS LDRLMNPLID QYLYYLNRTQ
451 NQSGSAQNKD LLFSRGSPAG MSVQPKNWLP GPCYRQQRVS KTKTDNNNSN
501 FTWTGASKYN LNGRESIINP GTAMASHKDD KDKFFPMSGV MIFGKESAGA
551 SNTALDNVMI TDEEEIKATN PVATERFGTV AVNLQSSSTD PATGDVHVMG
601 ALPGMVWQDR DVYLQGPIWA KIPHTDGHFH PSPLMGGFGL KHPPPQILIK
651 NTPVPANPPA EFSATKFASE ITQYSTGQVS VEIEWELQKE NSKRWNPEVQ
701 YTSNYAKSAN VDFTVDNNGL YTEPRPIGTR YLTRPL
Example of an amino acid sequence of wild-type AAV7 capsid protein
(SEQ ID NO: 22)
    1 MAADGYLPDW LEDNLSEGIR EWWDLKPGAP KPKANQQKQD NGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP AKKRPVEPSP
151 QRSPDSSTGI GKKGQQPARK RLNFGQTGDS ESVPDPQPLG EPPAAPSSVG
201 SGTVAAGGGA PMADNNEGAD GVGNASGNWH CDSTWLGDRV ITTSTRTWAL
251 PTYNNHLYKQ ISSETAGSTN DNTYFGYSTP WGYFDFNRFH CHFSPRDWQR
301 LINNNWGFRP KKLRFKLFNI QVKEVTTNDG VTTIANNLTS TIQVESDSEY
351 QLPYVLGSAH QGCLPPFPAD VFMIPQYGYL TLNNGSQSVG RSSFYCLEYF
401 PSQMLRTGNN FEFSYSFEDV PFHSSYAHSQ SLDRLMNPLI DQYLYYLART
451 QSNPGGTAGN RELQFYQGGP STMAEQAKNW LPGPCFRQQR VSKTLDQNNN
501 SNFAWTGATK YHLNGRNSLV NPGVAMATHK DDEDRFFPSS GVLIFGKTGA
551 TNKTTLENVL MTNEEEIRPT NPVATEEYGI VSSNLQAANT AAQTQVVNNQ
601 GALPGMVWQN RDVYLQGPIW AKIPHTDGNF HPSPLMGGFG LKHPPPQILI
651 KNTPVPANPP EVFTPAKFAS FITQYSTGQV SVEIEWELQK ENSKRWNPEI
701 QYTSNFEKQT GVDFAVDSQG VYSEPRPIGT RYLTRNL
Example of an amino acid sequence of wild-type AAV8 capsid protein
(SEQ ID NO: 23)
    1 MAADGYLPDW LEDNLSEGIR EWWALKPGAP KPKANQQKQD DGRGLVLPGY
  51 KYLGPFNGLD KGEPVNAADA AALEHDKAYD QQLQAGDNPY LRYNHADAEF
101 QERLQEDTSF GGNLGRAVFQ AKKRVLEPLG LVEEGAKTAP GKKRPVEPSP
151 QRSPDSSTGI GKKGQQPARK RLNFGQTGDS ESVPDPQPLG EPPAAPSGVG
201 PNTMAAGGGA PMADNNEGAD GVGSSSGNWH CDSTWLGDRV ITTSTRTWAL
251 PTYNNHLYKQ ISNGTSGGAT NDNTYFGYST PWGYFDFNRF HCHFSPRDWQ
301 RLINNNWGFR PKRLSFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFQFTYTFED VPFHSSYAHS QSLDRLMNPL IDQYLYYLSR
451 TQTTGGTANT QTLGFSQGGP NTMANQAKNW LPGPCYRQQR VSTTTGQNNN
501 SNFAWTAGTK YHLNGRNSLA NPGIAMATHK DDEERFFPSN GILIFGKQNA
551 ARDNADYSDV MLTSEEEIKT TNPVATEEYG IVADNLQQQN TAPQIGTVNS
601 QGALPGMVWQ NRDVYLQGPI WAKIPHTDGN FHPSPLMGGF GLKHPPPQIL
651 IKNTPVPADP PTTFNQSKLN SFITQYSTGQ VSVEIEWELQ KENSKRWNPE
701 IQYTSNYYKS TSVDFAVNTE GVYSEPRPIG TRYLTRNL
Example of an amino acid sequence of wild-type AAV9 capsid protein
(SEQ ID NO: 24)
    1 MAADGYLPDW LEDNLSEGIR EWWALKPGAP QPKANQQHQD NARGLVLPGY
  51 KYLGPGNGLD KGEPVNAADA AALEHDKAYD QQLKAGDNPY LKYNHADAEF
101 QERLKEDTSF GGNLGRAVFQ AKKRLLEPLG LVEEAAKTAP GKKRPVEQSP
151 QEPDSSAGIG KSGAQPAKKR LNFGQTGDTE SVPDPQPIGE PPAAPSGVGS
201 LTMASGGGAP VADNNEGADG VGSSSGNWHC DSQWLGDRVI TTSTRTWALP
251 TYNNHLYKQI SNSTSGGSSN DNAYFGYSTP WGYFDFNRFH CHFSPRDWQR
301 LINNNWGFRP KRLNFKLFNI QVKEVTDNNG VKTIANNLTS TVQVFTDSDY
351 QLPYVLGSAH EGCLPPFPAD VFMIPQYGYL TLNDGSQAVG RSSFYCLEYF
401 PSQMLRTGNN FQFSYEFENV PFHSSYAHSQ SLDRLMNPLI DQYLYYLSKT
451 INGSGQNQQT LKFSVAGPSN MAVQGRNYIP GPSYRQQRVS TTVTQNNNSE
501 FAWPGASSWA LNGRNSLMNP GPAMASHKEG EDRFFPLSGS LIFGKQGTGR
551 DNVDADKVMI TNEEEIKTIN PVATESYGQV ATNHQSAQAQ AQTGWVQNQG
601 ILPGMVWQDR DVYLQGPIWA KIPHTDGNFH PSPLMGGFGM KHPPPQILIK
651 NTPVPADPPT AFNKDKLNSF ITQYSTGQVS VEIEWELQKE NSKRWNPEIQ
701 YTSNYYKSNN VEFAVNTEGV YSEPRPIGTR YLTRNL
Example of an amino acid sequence of wild-type AAV10 capsid protein
(SEQ ID NO: 25)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGTSGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFEFSYQFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTFSQAKLA SFITQYSTGQ VSVEIEWELQ KENSKRWNPE
701 IQYTSNYYKS TNVDFAVNTD GTYSEPRPIG TRYLTRNL
Example of an amino acid sequence of wild-type AAV11 capsid protein
(SEQ ID NO: 26)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPLESPQ
151 EPDSSSGIGK KGKQPARKRL NFEEDTGAGD GPPEGSDTSA MSSDIEMRAA
201 PGGNAVDAGQ GSDGVGNASG DWHCDSTWSE GKVTTTSTRT WVLPTYNNHL
251 YLRLGTTSSS NTYNGFSTPW GYFDFNRFHC HFSPRDWQRL INNNWGLRPK
301 AMRVKIFNIQ VKEVTTSNGE TTVANNLIST VQIFADSSYE LPYVMDAGQE
351 GSLPPFPNDV FMVPQYGYCG IVTGENQNQT DRNAFYCLEY FPSQMLRTGN
401 NFEMAYNFEK VPFHSMYAHS QSLDRLMNPL LDQYLWHLQS TTSGETLNQG
451 NAATTFGKIR SGDFAFYRKN WLPGPCVKQQ RESKTASQNY KIPASGGNAL
501 LKYDTHYTLN NRWSNIAPGP PMATAGPSDG DESNAQLIFP GPSVTGNTTT
551 SANNLLFTSE EEIAATNPRD TDMFGQIADN NQNATTAPIT GNVTAMGVLP
601 GMVWQNRDIY YQGPIWAKIP HADGHFHPSP LIGGFGLKHP PPQIFIKNTP
651 VPANPATTFT AARVDSFITQ YSTGQVAVQI EWEIEKERSK RWNPEVQFTS
701 NYGNQSSMLW APDTTGKYTE PRVIGSRYLT NHL
Example of an amino acid sequence of wild-type AAV12 capsid protein
(SEQ ID NO: 27)
    1 MAADGYLPDW LEDNLSEGIR EWWALKPGAP QPKANQQHQD NGRGLVLPGY
  51 KYLGPFNGLD KGEPVNEADA AALEHDKAYD KQLEQGDNPY LKYNHADAEF
101 QQRLATDTSF GGNLGRAVFQ AKKRILEPLG LVEEGVKTAP GKKRPLEKTP
151 NRPTNPDSGK APAKKKQKDG EPADSARRTL DFEDSGAGDG PPEGSSSGEM
201 SHDAEMRAAP GGNAVEAGQG ADGVGNASGD WHCDSTWSEG RVTTTSTRTW
251 VLPTYNNHLY LRIGTTANSN TYNGFSTPWG YFDENRFHCH FSPRDWQRLI
301 NNNWGLRPKS MRVKIFNIQV KEVTTSNGET TVANNLTSTV QIFADSTYEL
351 PYVMDAGQEG SFPPFPNDVF MVPQYGYCGV VTGKNQNQTD RNAFYCLEYF
401 PSQMLRTGNN FEVSYQFEKV PFHSMYAHSQ SLDRMMNPLL DQYLWHLQST
451 TTGNSLNQGT ATTTYGKITT GDFAYYRKNW LPGACIKQQK FSKNANQNYK
501 IPASGGDALL KYDTHTTLNG RWSNMAPGPP MATAGAGDSD FSNSQLIFAG
551 PNPSGNTTTS SNNLLFTSEE EIATTNPRDT DMFGQIADNN QNATTAPHIA
601 NLDAMGIVPG MVWQNRDIYY QGPIWAKVPH TDGHFHPSPL MGGFGLKHPP
651 PQIFIKNTPV PANPNTTFSA ARINSFLTQY STGQVAVQID WEIQKEHSKR
701 WNPEVQFTSN YGTQNSMLWA PDNAGNYHEL RAIGSRFLTH HL
Example of an amino acid sequence of wild-type AAVrh10 capsid protein
(SEQ ID NO: 28)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGTSGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFEFSYQFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTFSQAKLA SFITQYSTGQ VSVEIEWELQ KENSKRWNPE
701 IQYTSNYYKS TNVDFAVNTD GTYSEPRPIG TRYLTRNL
Example of a nucleotide sequence encoding AAVrh74 capsid protein:
(SEQ ID NO: 29)
atggctgccgatggttatcttccagattggctcgaggacaacctctctgagggcattcgcgagtggtgggacctgaa
acctggagccccgaaacccaaagccaaccagcaaaagcaggacaacggccggggtctggtgcttcctggctacaagt
acctcggacccttcaacggactcgacaagggggagcccgtcaacgcggcggacgcagcggccctcgagcacgacaag
gcctacgaccagcagctccaagcgggtgacaatccgtacctgcggtataatcacgccgacgccgagtttcaggagcg
tctgcaagaagatacgtcttttgggggcaacctcgggcgcgcagtcttccaggccaaaaagcgggttctcgaacctc
tgggcctggttgaatcgccggttaagacggctcctggaaagaagagaccggtagagccatcaccccagcgctctcca
gactcctctacgggcatcggcaagaaaggccagcagcccgcaaaaaagagactcaattttgggcagactggcgactc
agagtcagtccccgaccctcaaccaatcggagaaccaccagcaggcccctctggtctgggatctggtacaatggctg
caggcggtggcgctccaatggcagacaataacgaaggcgccgacggagtgggtagttcctcaggaaattggcattgc
gattccacatggctgggcgacagagtcatcaccaccagcacccgcacctgggccctgcccacctacaacaaccacct
ctacaagcaaatctccaacgggacctcgggaggaagcaccaacgacaacacctacttcggctacagcaccccctggg
ggtattttgacttcaacagattccactgccacttttcaccacgtgactggcagcgactcatcaacaacaactgggga
ttccggcccaagaggctcaacttcaagctcttcaacatccaagtcaaggaggtcacgcagaatgaaggcaccaagac
catcgccaataaccttaccagcacgattcaggtctttacggactcggaataccagctcccgtacgtgctcggctcgg
cgcaccagggctgcctgcctccgttcccggcggacgtcttcatgattcctcagtacgggtacctgactctgaacaat
ggcagtcaggctgtgggccggtcgtccttctactgcctggagtactttccttctcaaatgctgagaacgggcaacaa
ctttgaattcagctacaacttcgaggacgtgcccttccacagcagctacgcgcacagccagagcctggaccggctga
tgaaccctctcatcgaccagtacttgtactacctgtcccggactcaaagcacgggcggtactgcaggaactcagcag
ttgctattttctcaggccgggcctaacaacatgtcggctcaggccaagaactggctacccggtccctgctaccggca
gcaacgcgtctccacgacactgtcgcagaacaacaacagcaactttgcctggacgggtgccaccaagtatcatctga
atggcagagactctctggtgaatcctggcgttgccatggctacccacaaggacgacgaagagcgattttttccatcc
agcggagtcttaatgtttgggaaacagggagctggaaaagacaacgtggactatagcagcgtgatgctaaccagcga
ggaagaaataaagaccaccaacccagtggccacagaacagtacggcgtggtggccgataacctgcaacagcaaaacg
ccgctcctattgtaggggccgtcaatagtcaaggagccttacctggcatggtgtggcagaaccgggacgtgtacctg
cagggtcccatctgggccaagattcctcatacggacggcaactttcatccctcgccgctgatgggaggctttggact
gaagcatccgcctcctcagatcctgattaaaaacacacctgttcccgccgatcctccgaccaccttcaatcaggcca
agctggcttctttcatcacgcagtacagtaccggtcaggtcagcgtggagatcgagtgggagctgcagaaggagaac
agcaaacgctggaacccagagattcagtacacttccaactactacaaatctacaaatgtggactttgctgtcaatac
tgagggtacttattccgagcctcgccccattggcacccgttacctcacccgtaatctgtaa
```

### Nucleic Acids Encoding AAV Capsid Proteins

Provided herein are nucleic acids encoding capsid proteins. A nucleic acid may comprise a sequence that encodes a capsid protein disclosed here that comprises a wild-type amino acid sequence or a capsid protein comprising one or more amino acid substitutions. A sequence encoding a capsid protein disclosed herein can be determined by one of ordinary skill in the art by known methods. A nucleic acid encoding a capsid protein may comprise a promoter or other regulatory sequence operably linked to the coding sequence. A nucleic acid encoding a capsid protein may be in the form of a plasmid, an mRNA, or another nucleic acid capable of being used by enzymes or machinery of a host cell to produce a capsid protein. Nucleic acids encoding capsid proteins as provided herein can be used to make AAV particles that can be used for delivering a gene to a cell. Methods of making AAV particles are known in the art. For example, see Scientific Reports volume 9, Article number: 13601 (2019); Methods Mol Biol. 2012; 798:267-284; and www.thermofisher.com/us/en/home/clinical/cell-gene-therapy/gene-therapy/aav-production-workflow.html.

### Transduction Efficiency

In some embodiments, the AAV particles comprising a nucleic acid vector comprising an ITR comprising a MyoD binding site and/or MEF binding site has a higher transduction efficiency compared to a corresponding wild-type AAV of the same serotype or a corresponding AAV not comprising the MyoD binding site and/or MEF binding site. Transduction efficiency of an AAV particle can be determined, for example, by comparing expression of a transgene in a cell following contacting the cell with the AAV particle. In some embodiments, transduction efficiency of an AAV particle as disclosed herein (e.g., an AAV particle comprising an ITR comprising a MyoD binding site and/or MEF binding site) is higher than the transduction efficiency of a corresponding wild-type AAV particle or of an AAV particle of the same serotype but which does not have the MyoD binding site and/or MEF binding site. In some embodiments, the transduction efficiency of an AAV particle as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the transduction efficiency of a corresponding wild-type AAV particle or of an AAV particle of the same serotype but which does not have the MyoD binding site and/or MEF binding site. In some embodiments, the transduction efficiency of an AAV particle as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the transduction efficiency of a corresponding wild-type AAV particle or of an AAV particle of the same serotype but which does not have the MyoD binding site and/or MEF binding site.

### Pharmaceutical Compositions

Any one of the AAV particles disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Use

According to some aspects, methods of contacting a cell with an AAV particle are provided herein. Methods of contacting a cell may comprise, for example, contacting a cell in a culture with a composition comprising an AAV particle. In some embodiments, contacting a cell comprises adding a composition comprising an AAV particle to the supernatant of a cell culture (e.g., a cell culture on a tissue culture plate or dish) or mixing a composition comprising an AAV particle with a cell culture (e.g., a suspension cell culture). In some embodiments, contacting a cell comprises mixing a composition comprising an AAV particle with another solution, such as a cell culture media, and incubating a cell with the mixture.

In some embodiments, contacting a cell with an AAV particle comprises administering a composition comprising an AAV particle to a subject or device in which the cell is located. In some embodiments, contacting a cell comprises injecting a composition comprising an AAV particle into a subject in which the cell is located. In some embodiments, contacting a cell comprises administering a composition comprising an AAV particle directly to a cell, or into or substantially adjacent to a tissue of a subject in which the cell is present.

Aspects of this disclosure provide a method comprising administering to a subject any one of the compositions comprising any one of the AAV particles disclosed herein.

In some embodiments, “administering” or “administration” means providing a material to a subject in a manner that is pharmacologically useful. In some embodiments, an AAV particle (e.g., comprised in a composition) is administered to a subject enterally. In some embodiments, an enteral administration of the essential metal element/s is oral. In some embodiments, an AAV particle is administered to the subject parenterally. In some embodiments, an AAV particle is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, an AAV particle is administered to the subject by injection into the hepatic artery or portal vein.

In some embodiments, a composition of AAV particles is administered to a subject to treat a disease or condition. To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., a muscular dystrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a composition comprising any one of the particles disclosed herein comprises at least 2 times (e.g., 2-200 times, 2-4 times, 2-10 times, 5-10 times, 2-20 times, 10-20 times, 10-50 times, 20-50 times, 50-100 times, 50-200 times or more) less AAV particles compared to a composition of wild-type AAV particles would have to be to achieve the same transgene expression in the same cells/tissue. For example, if 10¹⁴ particles of a wild-type AAVrh74 particle with ITRs not comprising a MyoD binding site and/or MEF binding site would have to be administered to achieve express a certain level of transgene in muscle tissue, then less than 10¹⁴ particles (e.g., 10¹³ particles or 10¹² particles) comprising an ITR comprising a MyoD binding site and/or MEF binding site would have to be administered. In some embodiments, the amount of AAV particles comprising a MyoD binding site and/or MEF binding site in an ITR needed to achieve the same level of transgene expression as an AAV particle without the MyoD binding site and/or MEF binding site is at least 10% less than that of the particle without the MyoD binding site and/or MEF binding site.

In some embodiments, a cell disclosed herein is a cell isolated or derived from a subject. In some embodiments, a cell is a mammalian cell (e.g., a cell isolated or derived from a mammal). In some embodiments, a cell is a human cell. In some embodiments, a cell is isolated or derived from a particular tissue of a subject, such as muscle tissue. In some embodiments, a cell is a muscle cell. In some embodiments, a cell is a skeletal muscle cell or a smooth muscle cell. In some embodiments, a cell is in vitro. In some embodiments, a cell is ex vivo. In some embodiments, a cell is in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

In some embodiments, the concentration of AAV particles administered to a subject may be on the order ranging from 10⁶ to 10¹⁵ particles/ml or 10³ to 10¹⁶ particles/ml, or any values therebetween for either range, such as for example, about 10⁶, 10⁷, 10⁸, 10⁹, 10¹⁰, 10¹¹, 10¹², 10¹³, 10¹⁴ or 10¹⁵ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of AAV (e.g., AAVrh74) composition delivered to a subject (e.g., via one or more routes of administration as described herein) is 0.0001 ml to 10 ml.

In some embodiments, a composition disclosed herein (e.g., comprising an AAV particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

### Subjects

Aspects of the disclosure relate to methods for use with a subject, such as human or non-human primate subjects; with a host cell in situ in a subject; or with a host cell derived from a subject (e.g., ex vivo or in vitro). Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease or disorder that may be treated with gene therapy. In some embodiments, the subject has or is suspected of having a muscle disease or disorder. A muscle disease or disorder is typically characterized by one or more mutation(s) in the genome that results in abnormal structure or function of one or more proteins associated with muscle development, health, maintenance and/or function. Exemplary muscle disease and disorders include amyotrophic lateral sclerosis, Charcot-Marie-Tooth disease, multiple sclerosis, muscular dystrophy (e.g., Duchenne muscular dystrophy, facioscapulohumeral muscular dystrophy, Becker muscular dystrophy, or limb-girdle muscular dystrophy (LGMD) such as LGMD type 1 or LGMD type 2), myasthenia gravis, myopathy (e.g., X-linked myotubular myopathy), myositis, peripheral neuropathy, or spinal muscular atrophy. Muscle diseases and disorders can be characterized and identified, e.g., through laboratory tests and/or evaluation by a clinician. In some embodiments, the subject has or is suspected of having a disease involving muscle cells (e.g., a disease caused by a defect, such as a genetic mutation, in one or more muscle cells or genes associated therewith). In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with muscle development, health, maintenance, or function).

In some embodiments, a gene associated with muscle development, health, maintenance, or function is dystrophin/DMD, SCN4A, DMPK, ACTA, TPM3, TPM2, TNNT1, CFL2, KBTBD13, KLHL30, KKLHL3, KLHL41, LMOD3, MYPN, MTM1, nebulin, DNM2, TTN, RYR1, MYH7, TK2, GAA (α-glucosidase), ClC1, LMNA, CAV3, DNAJB6, TRIM32, desmin, LAMA2, COL6A1, COL6A2, COL6A3, or DUX4. In some embodiments the gene is dystrophin (DMD) or MTM1. In some embodiments, the gene is a gene in which mutations have been shown to cause limb-girdle muscular dystrophy (e.g., LGMD1 or LGMD2), such as MYOT, LMNA, CAV3, DNAJB6, DES, TNP03, HNRNPDL, CAPN3, DYSF, SGCG, SGCA, SGCB, SGCD, TCAP. TRIM32, FKRP, TTN, POMT1, ANO5, FKTN, POMT2, POMGnT1, DAG1, PLEC1, DES, TRAPPC11, GMPPB, ISPD, GAA, LIMS2, BVES, or TOR1A1P1. In some embodiments, a subject comprises a mutant form of one or more genes associated with muscle development, health, maintenance or function. In some embodiments, methods disclosed herein provide a cell (e.g., a muscle cell) of a subject with a functional form of a gene associated with muscle development, health, maintenance, or function.

### EXAMPLES

### Example 1: Strategy to Improve Transgene Expression of a Transgene Delivered Using AAV Particles in Muscle Cells by Modification of AAV ITR

FIGS. 2A and 2B provide examples of ITRs comprising a MyoD, and MyoD and MEF binding sites, respectively. The level of transgene expression in a muscle cell using an AAV particle comprising an ITR comprising a MyoD binding site (as shown in FIG. 2A) is higher than the level of transgene expression in a muscle cells using an AAV particle comprising an ITR not comprising a MyoD binding site. The level of transgene expression in a muscle cell using an AAV particle comprising an ITR comprising a MEF binding site is higher than the level of transgene expression in a muscle cells using an AAV particle comprising an ITR not comprising a MEF binding site. The level of transgene expression in a muscle cell using an AAV particle comprising an ITR comprising a MyoD binding site and a MEF binding site (as shown in FIG. 2B) is higher than the level of transgene expression in a muscle cell using an AAV particle comprising an ITR not comprising a MyoD binding site, or an AAV particle comprising an ITR comprising only a MyoD binding sitc.

### Other Embodiments

All of the features disclosed in this specification may be combined in any combination. Each feature disclosed in this specification may be replaced by an alternative feature serving the same, equivalent, or similar purpose. Thus, unless expressly stated otherwise, cach feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An adeno-associated virus (AAV) particle comprising a nucleic acid vector, wherein the nucleic acid vector comprises a 5′ inverted terminal repeat (ITR) comprising a myoblast determination protein (MyoD) binding site and/or myocyte enhancer factor (MEF) binding site.

2. An AAV particle of claim 1, wherein the 5′ ITR comprises a MyoD binding site.

3. The AAV particle of claim 1 or 2, wherein the MyoD binding site comprises the nucleic acid sequence 5′-AGCAGCTGCT-3′ (SEQ ID NO: 1).

4. The AAV particle of claim 1 or 2, wherein the MyoD binding site comprises the nucleic acid sequence 5′-TCGTCGACG-3′ or 5′-AGCAGCTGC-3′.

5. An AAV particle of any one of claims 1 to 4, wherein the 5′ ITR comprises a MEF binding site.

6. The AAV particle of claim 1 or 5, wherein the MEF binding site comprises the nucleic acid sequence 5′-CTAAAAATAG-3′ (SEQ ID NO: 4).

7. The AAV particle of claim 1 or 5, wherein the MEF binding site comprises the nucleic acid sequence 5′-GATTTTTATC-3′ (SEQ ID NO: 33).

8. The AAV particle of any one of claims 1 to 7, wherein the 5′ ITR comprises a MyoD binding site and a MEF binding site, optionally wherein the MEF binding site is upstream of (5′ relative to) the MyoD binding site.

9. The AAV particle of any one of claims 1 to 8, wherein the MyoD and/or MEF binding sites are comprised upstream from (5′ relative to) the terminal resolution site of the ITR.

10. The AAV particle of any one of claims 1 to 9, wherein the nucleic acid vector further comprises a transgene, and optionally a muscle-specific promoter.

11. The AAV particle of any one of claims 1 to 10, wherein the AAV particle is an AAVrh74 particle.

12. The AAV particle of any one of claims 1 to 11, wherein the nucleic acid vector is of serotype 2.

13. A composition comprising the AAV particle of any one of claims 1 to 12 and a pharmaceutically acceptable carrier.

14. A method comprising delivering to a cell or administering to a subject the AAV particle of any one of claims 1-12 or the composition of claim 13.

15. The method of claim 14, wherein the cell is a human muscle cell or the subject is human.

16. The method of claim 14 or 15, wherein the transduction efficiency of the AAV particle of any one of claims 1-12 is at least 2 times higher than the transduction efficiency of an AAV particle comprising a 5′ ITR lacking MyoD and/or MEF binding sites.
